(19) United States
(12) Patent Application Publication
(10) Pub. No.: US 2022/0088238 A1
(43) Pub. Date: Mar. 24, 2022

(54) ULTRAVIOLET SANITIZATION UNIT FOR BEVERAGE CUPS

(71) Applicant: ValidFill LLC, Bradenton, FL (US)

(72) Inventor: Jeremy Wade, Bradenton, FL (US)

(21) Appl. No.: 17/225,522

(22) Filed: Apr. 8, 2021

Related U.S. Application Data
(60) Provisional application No. 63/199,135, filed on Dec. 9, 2020.
(60) Provisional application No. 63/007,036, filed on Apr. 8, 2020.

(51) Int. Cl. A61L 2/10; B67C 7/00

(52) U.S. Cl. CPC A61L 2/10; B67C 7/00; A61L 2202/23; A61L 2202/14; A61L 2202/121; A61L 2202/122; A61L 2202/11

(57) ABSTRACT

The system relates to an ultraviolet (UV) sanitization unit configured to detect and receive a beverage cup with a cradle portion of the unit. Once detected, the unit will shutter a door to prevent UV light from exiting the cradle portion and causing harm to users. Inside the cradle includes a plurality of UV emitter assemblies that are configured to continuously emit UV light when the unit is activated but to shutter the light from radiating into the cradle portion until the beverage cup is placed within the cradle. The beverage cup is placed in the cradle portion and the door is shuttered. The UV emitter assembly shutters are rotated to allow the UV light from the UV emitter assemblies to be incident upon the beverage cup. After irradiation for a predetermined time, the UV emitter assembly shutters are rotated to shutter the UV emitter assemblies, the door is un-shuttered, and the user removes the sanitized beverage cup.

### CROSS-REFERENCE TO RELATED APPLICATIONS

This application is related to and claims the benefit of U.S. Provisional Application No. 63/007,036, filed on Apr. 8, 2020, and U.S. Provisional Application No. 63/199,135, filed on Dec. 9, 2020, the entire contents of each being incorporated herein by reference.

### FIELD OF THE INVENTION

The system relates to an ultraviolet (UV) sanitization unit configured to detect and receive a beverage container with a cradle portion of the unit, wherein upon detection UV light is caused to be incident upon the beverage container to sanitize it.

### BACKGROUND OF THE INVENTION

Refillable containers is a huge part of sustainability programs globally and needs to be utilized to minimize waste. These containers are utilized for beverage dispensing which includes coffee, soda, beer, still beverages, cannabidiol (cbd) based beverages, etc. Transfer of viruses and bacteria from a beverage container to a dispenser, then from the dispenser to another container is a public health concern. Virus and bacteria transfer can occur when contact of the container is made to the dispensing lever or when the container contacts the dispensing valve.

Even when contact with a dispenser is not made, viruses and bacteria can live on the beverage container, posing a risk to the end user. Sanitization of these containers is needed at or near the point of beverage dispensing to minimize the risk to the users.

### SUMMARY OF THE INVENTION

The system relates to an ultraviolet (UV) sanitization unit configured to detect and receive a beverage container with a cradle portion of the unit. Once detected, the unit will shutter a door to prevent UV light from exiting the cradle portion and causing harm to users. Inside the cradle portion includes a plurality of UV emitter assemblies that are configured to continuously emit UV light when the unit is activated but to shutter the light from radiating into the cradle portion until the beverage container is placed within the cradle portion of the unit. In an exemplary use, the sanitization unit is activated to cause the UV emitter assemblies to emit UV light but said assemblies are shuttered by UV emitter assembly shutters. A user places a beverage container in the cradle portion and the door is shuttered to protect the user. The UV emitter assembly shutters are rotated to allow the UV light from the UV emitter assemblies to be incident upon the beverage container within the cradle portion. After irradiation for a predetermined time (e.g., 20 seconds), the UV emitter assembly shutters are actuated to shutter the UV emitter assemblies, the door is un-shuttered, and the user removes the sanitized beverage cup.

In an exemplary embodiment, a sanitization unit includes a housing having at least one ultraviolet emitter assembly, the housing configured to have a cradle portion to receive an object. The unit includes a door comprising a material that is transparent or translucent to light in the visible spectrum but blocks light in the ultraviolet spectrum, the door further comprising an actuator to open and close the door. The unit includes a processor in communication with a sensor, wherein: the sensor detects presence or proximity of the object, and emits a signal; the processor receives the signal and, upon receipt of said signal, automatically actuates the door actuator to grant access to the cradle portion.

Some embodiments include a rotating plate upon which the object is placed when placed within the housing, the rotating plate further comprising an actuator to rotate the plate.

Some embodiments include a sensor to detect when the object is placed on the plate, wherein when detection occurs the sensor emits a signal. The processor receives the signal and, upon receipt of said signal, automatically actuates the plate actuator to rotate the plate.

In some embodiments, the at least one ultraviolet emitter assembly comprises an ultraviolet lamp.

In some embodiments, the at least one ultraviolet emitter assembly comprises a shroud configured to selectively shutter the at least one ultraviolet emitter assembly.

In some embodiments, upon activation of the sanitization unit, the at least one ultraviolet emitter assembly continuously emits ultraviolet light.

Some embodiments include a sensor to detect when the object is placed within the cradle portion, wherein when detection occurs the sensor emits a signal. The at least one ultraviolet emitter assembly comprises a shroud configured to selectively shutter the at least one ultraviolet emitter assembly. Upon activation of the sanitization unit, the at least one ultraviolet emitter assembly continuously emits ultraviolet light. When the processor receives the signal, the processor causes the shroud for the at least one ultraviolet emitter assembly to automatically un-shutter the at least one ultraviolet emitter assembly.

Some embodiments include a sensor to detect shape, color, material composition, and/or orientation of the object.

Some embodiments include a sensor to configured as a RFID reader, wherein the object includes a RFID tag.

Some embodiments include a sanitization sensor.

Some embodiments include an ultraviolet light environment sensor.

In some embodiments, the door includes an electro-mechanical lock.

In some embodiments, the door includes an electro-mechanical lock. The processor is in connection with first ultraviolet light environment sensor and the electro-mechanical lock is in connection with a second ultraviolet light environment sensor.

Some embodiments include a display.

In some embodiments, the display is configured to generate a user interface that facilitates command and control of the sanitization unit.

Some embodiments include a display, and a sanitization sensor, wherein the sanitization sensor generates sanitization data that is displayed via a user interface of the display.

An exemplary sanitization and dispensing system includes sanitization unit configured to receive a beverage container and sanitize said beverage container via ultraviolet radiation. The system includes a beverage dispensing unit configured to disburse beverage. The sanitization unit is in communication with the beverage dispensing unit.

In some embodiments, the sanitization unit tracks sanitization data pertaining to the beverage container and transmits said sanitization data to the beverage dispensing unit.

In some embodiments, the beverage dispensing unit does not disburse beverage unless said sanitization data indicates that the beverage container has been sanitized.

In some embodiments, the beverage dispensing unit does not disburse beverage unless said sanitization data indicates that the beverage container had been sanitized within a predetermine time before the beverage dispensing unit receives said sanitization data.

Further features, aspects, objects, advantages, and possible applications of the present invention will become apparent from a study of the exemplary embodiments and examples described below, in combination with the Figures.

### BRIEF DESCRIPTION OF THE DRAWINGS

The above and other objects, aspects, features, advantages and possible applications of the present invention will be more apparent from the following more particular description thereof, presented in conjunction with the following drawings, in which:

FIG. 1 shows an exploded view of an embodiment of the ultraviolet (UV) sanitization unit.

FIGS. 2-3 show various views of an embodiment of the UV sanitization unit.

FIGS. 4-7 show various exploded views of a cradle portion of the UV sanitization unit.

FIG. 8 shows a top portion of the cradle portion.

FIG. 9 shows an embodiment of UV emitter assembly.

FIG. 10 shows an exemplary system architecture block diagram for an embodiment of the UV sanitization unit.

FIG. 11-13 show exemplary flow diagrams for operating embodiments of the UV sanitization unit.

### DETAILED DESCRIPTION OF THE INVENTION

The following description is of an embodiment presently contemplated for carrying out the present invention. This description is not to be taken in a limiting sense, but is made merely for the purpose of describing the general principles and features of the present invention. The scope of the present invention should be determined with reference to the claims.

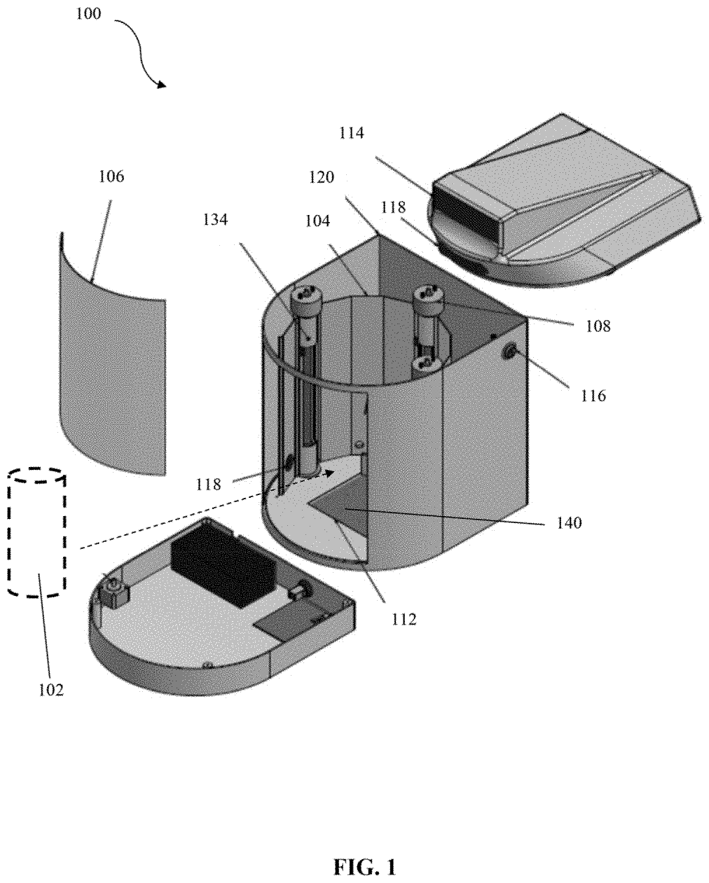

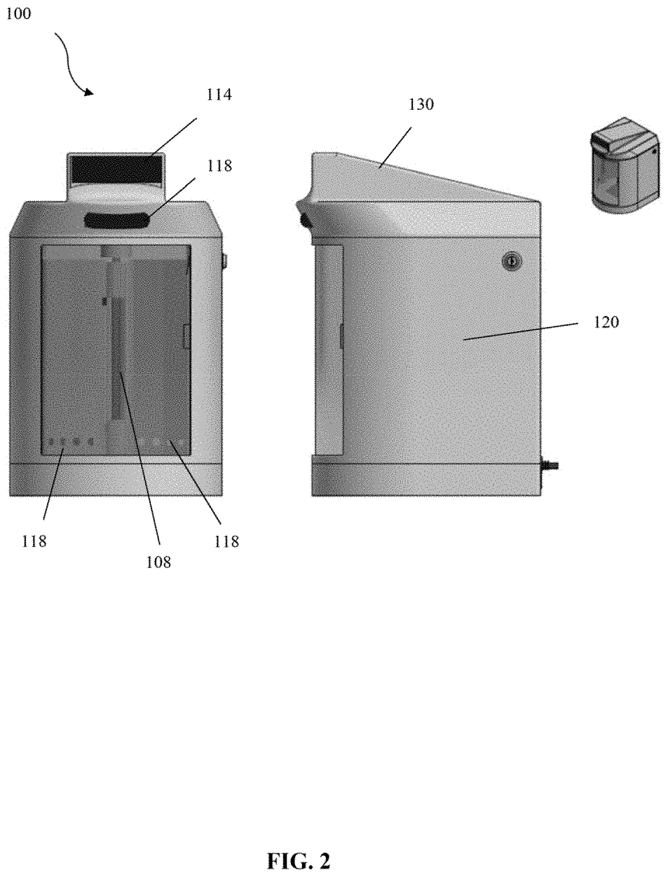

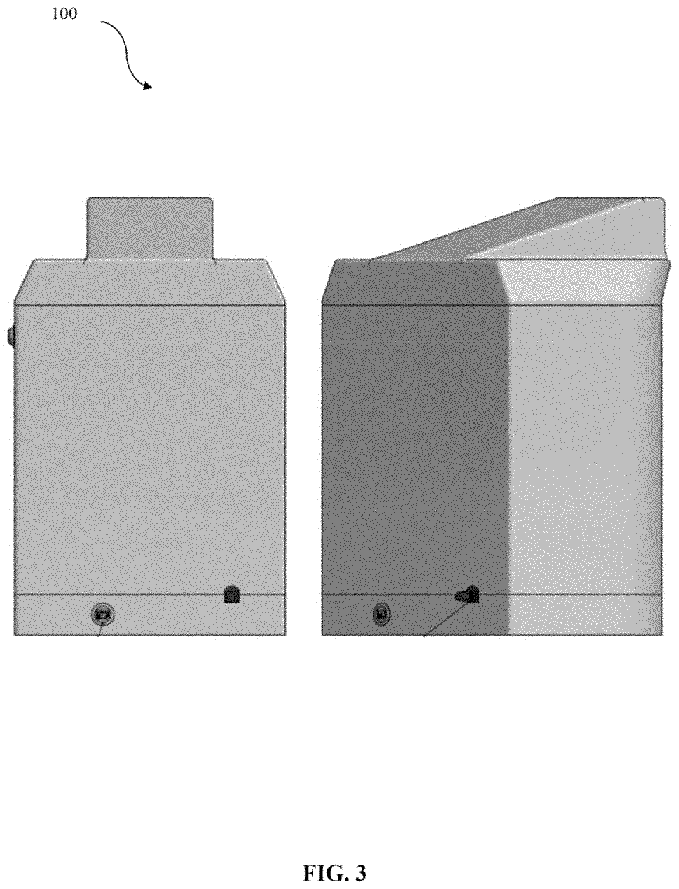

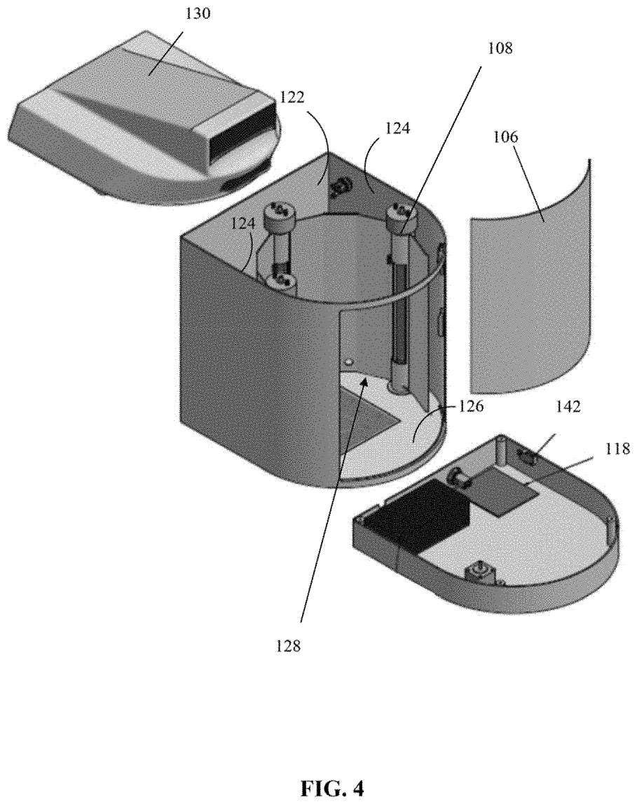

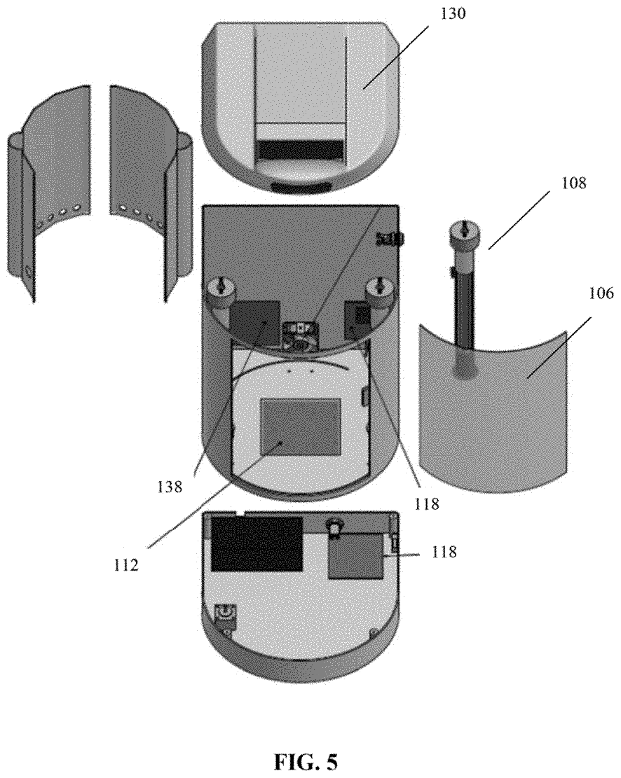

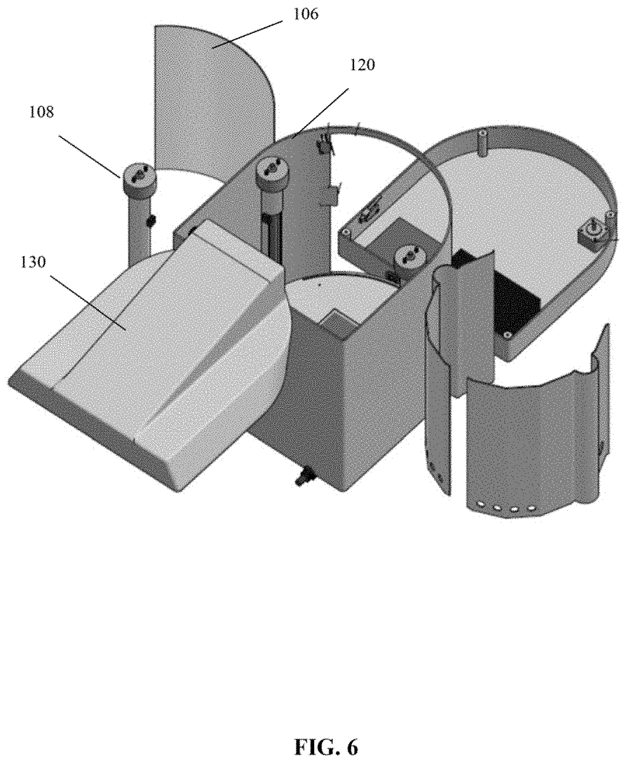

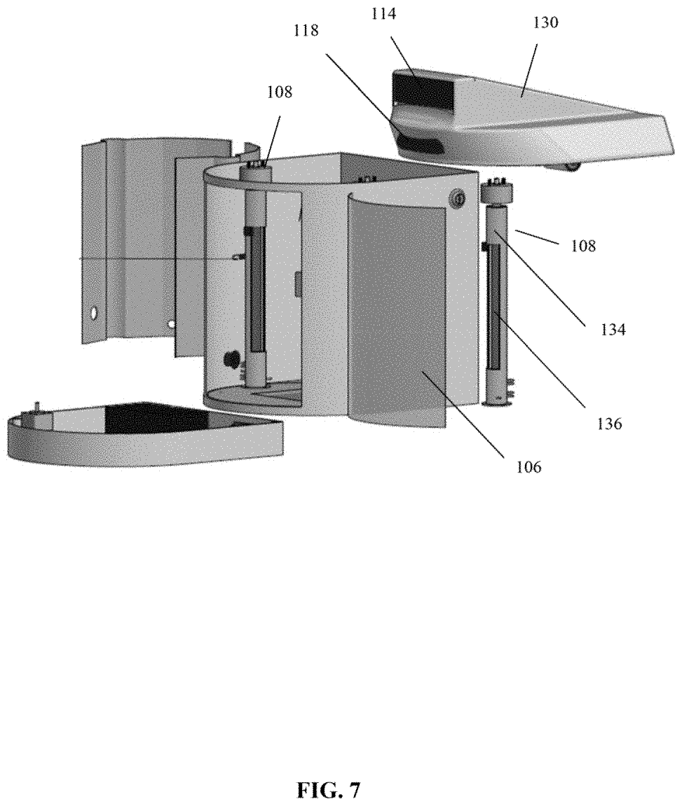

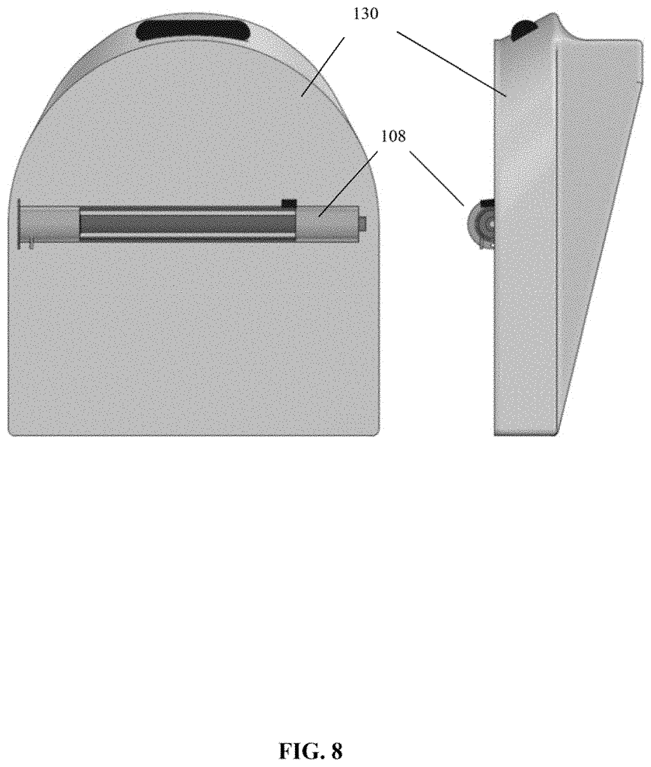

Referring to FIGS. 1-8, embodiments of the system relate to an ultraviolet (UV) sanitization unit 100 configured to detect and receive a beverage container 102 with a cradle portion 104 of the sanitization unit 100. Once detected, the sanitization unit 100 will shutter a door 106 to prevent UV light from exiting the cradle portion 104, thereby preventing causing harm to users. Inside the cradle portion 104 includes a plurality of UV emitter assemblies 108 that are configured to continuously emit UV light when the sanitization unit 100 is activated but to shutter the individual UV emitter assemblies 108 so as to prevent UV light from radiating into the cradle portion 104 until the beverage container 102 is placed within the cradle portion 104. In an exemplary use, the sanitization unit 100 is activated to cause the UV emitter assemblies 108 to emit UV light but said assemblies 108 are shuttered by UV emitter assembly shutters 110. A user places a beverage container 102 (or any other device for which sanitization via the unit 100 is desired) in the cradle portion 104 and the door 106 is shuttered to protect the user. The cradle portion 104 includes a plate 112 or platform upon which the beverage container 102 is placed. The plate 112 is in mechanical connection with a motor that causes the plate 112 to rotate, thereby causing the beverage container 102 to rotate. Thus, after the door 106 is shuttered, the motor is activated to cause the beverage container 102 to rotate. The UV emitter assembly shutters 110 are then rotated to allow the UV light from the UV emitter assemblies 108 to be incident upon the beverage container 102 within the cradle portion 104 as the beverage container 102 is rotated via the rotating plate 112. After irradiation for a predetermined time, the UV emitter assembly shutters 110 are actuated to shutter the individual UV emitter assemblies 108, the plate 112 stops rotating, the door 106 is un-shuttered, and the user removes the sanitized beverage container 102. The predetermined time is contemplated to be 20 seconds, but other times can be used. It is contemplated for each of the operational steps (e.g., shuttering and un-shuttering of the door 106 and shutters 110, starting and stopping the rotation of the plate 112, etc.) to occur automatically by the sanitization unit 100. However, some embodiments may include control features (actuated via a user interface of a display 114) to allow a user to control any one or combination of features.

It is contemplated for the UV sanitization unit 100 to have an on/off switch 116 to facilitate supply of electrical power to the sanitization unit 100. Once activated, electrical power is supplied to the UV emitter assemblies 108 causing them emit UV light continuously, as opposed to having a switch that activates the UV emitter assemblies 108 independent of the on/off switch 116 for the entire sanitization unit 100. Thus, once the sanitization unit 100 is turned on, the UV emitter assemblies 108 emit UV light. Only when the sanitization unit 100 is turned off will the UV emitter assemblies 108 not emit light. As will be explained herein, the UV emitter assemblies 108 have UV shutters 110 that shield UV light from being radiated into the cradle portion 104. These UV shutters 110 have a default position that is a closed (or shuttered) position. Then before the sanitization unit 100 is turned on and while the sanitization unit 100 is turned on, the UV emitter assembly shutters 110 are shuttered to prevent UV light from being emitted into the cradle portion 104, unless and until a beverage container 102 is placed within the cradle portion 104 (as detected via sensors 118), after which the door 106 is shuttered. Only then will the UV emitter assembly shutters 110 be activated and un-shutter the UV emitter assemblies 108 to permit UV light to enter into the cradle portion 104.

Having the UV emitter assemblies 108 “on” and emitting UV light continuously when the sanitization unit 100 is turned on (as opposed to having a separate on/off switch for the UV emitter assemblies 108) can be done to avoid operating delays. For instance, in order to meet the throughput demands in a retail environment (typically one consumer after another), it may not be desirable to turn the UV emitter assemblies 108 on and off for each use, as the reboot time to get each UV emitter assembly 108 back up to maximum dosage from an off position can be as much as one minute. Thus, shuttering UV emitter assemblies 108 that are left on as long as the sanitization unit 100 is turned on meets such throughput demand. However, the sanitization unit 100 can be configured to have a separate and independently controlled on/off switch for any one or combination of UV emitter assemblies 108. In addition, any one or combination of UV emitter assemblies 108 can be configured to radiate UV light continuously, periodically, in accordance to a predetermined pattern, etc. Any one or combination of UV emitter assemblies 108 can emit UV light at an intensity that differs from another UV emitter assembly 108, can be tunable so as to vary the intensity of UV light being emitted (which can be controlled via the processor 138), can be configured to strobe, etc.

The UV sanitization unit 100 has a housing 120, the housing 120 having a back wall 122, two side walls 124, a bottom surface 126, and a front opening 128. The shape of the housing 120 forms an interior cavity between the back wall 122, two side walls 124, bottom surface 126, and front opening 128. The interior cavity serves as a cradle portion 104 within which the beverage container 102 is placed for sanitization. The housing 120 includes a top 130. The top 130 can be removable so as to allow access to the cradle portion 104 and UV emitter assemblies 108 for maintenance purposes.

The interior cavity has at least one UV emitter assembly 108. The UV emitter assembly 108 includes at least one UV emitting germicidal lamp 132 (e.g., UV bulb, UV mercury lamp, UV excimer lamp, UV LED, etc.) configured to emit UV light. It may be preferred for the UV emitter assembly 108 to use a UV bulb as the UV lamp 132, as the costs associated with and the heat generated using UV LEDs may be undesirable.

UV light includes wavelengths that fall between visible light wavelengths and x-ray wavelengths on the electromagnetic spectrum—e.g., between 10 nanometers (nm) to 400 nm. The UV light range can be further divided into UV-A, UV-B, UV-C, and Vacuum-UV. Ultraviolet germicidal irradiation (UVGI) uses short wavelength ultraviolet light (UVC light or UV-C light) to kill microbes. The UV-C portion represents wavelengths from 200 nm to 280 nm. UV-C photons are able to penetrate cells and damage the nucleic acid, rendering them incapable of reproduction. When UV-C photons are imparted on a microbe, such damage to the nucleic acid causes them to microbiologically inactive. UV-C photons directed at viruses, bacteria, or other pathogens (regardless of them being in water, air, or on a surface of an object) renders these pathogens harmless in seconds. Thus, it is contemplated for the UV emitting germicidal lamp to emit light having wavelengths within a range from 200 nm to 280 nm. The most effective wavelengths against microbes are within the range of 260 nm to 275 nm, providing effective and consistent anti-microbial performance across a wide range of pathogenic organisms. Thus, it is contemplated for at least one of the UV emitting germicidal lamps to emit light having a wavelength within the range of 260 nm to 275 nm.

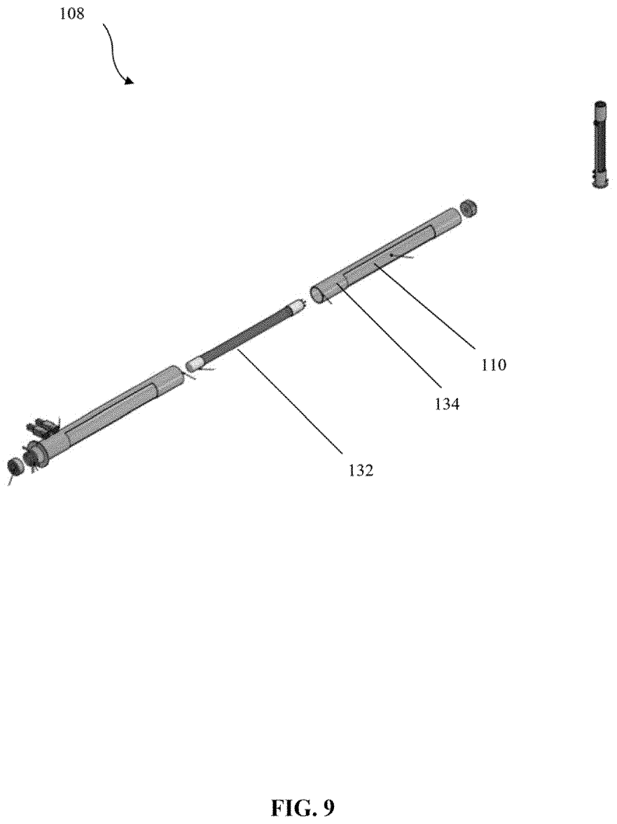

Each UV lamp 132 can be placed within a shroud 134. (See FIG. 9). For instance, the UV lamp 132 can be placed within a cylindrical shroud 134 attached to an inner surface of the housing 120. The shroud 134 has a shutter 110 that rotates to selectively expose the UV lamp 132. When un-shuttered, and the UV lamp 132 is emitting UV light, the UV lamp 132 is able emit UV light within the cavity, and preferably towards the center of the cavity where a beverage container 102 would be placed.

In an exemplary embodiment, each UV emitter assembly 108 includes an elongated UV lamp 132. The UV lamp 132 operates in the UV-C range. The UV lamp 132 is placed within a shroud 134. The shroud 134 is a cylindrical (e.g. tubular) member made of UV blocking material (e.g., plastic, ceramic, metal, etc.). The shroud 134 has a window 136 that is an opening formed within a side portion of the shroud 134. The shroud 134 physically contains the UV lamp 132 and contains any UV light emitted from the UV lamp 132 within the shroud 134 except for the window 136. On the shroud 134 is a shutter 110. The shutter 110 is a member that complements the contour of the shroud 134 and is configured to slide or rotate over the window 136 and away from the window 136. For instance, the shutter 110 can be a member fabricated from the same material as that of the shroud 134 and placed on a track or rail assembly, a bearing assembly, etc. attached to the shroud 134. Electrical motors, solenoid rotors, gearing, encoders, etc. can be used to facilitate motion of the shutter 110. When the shutter 110 is actuate to be over the window 136, no or very little UV light can escape the shroud 134, thereby shuttering the UV emitter assembly 108. When the shutter 110 is actuated to be away from the window 136, UV light from the UV lamp 132 escapes the shroud 134 and radiates from the window 136. Each shutter 110 can have a biasing-mechanism to bias the shutter 110 in a shuttered position. Thus, when the power is turned off or in the event of a power outage, the shutters 110 automatically shutter the UV lamps 132. In addition, the motor assembly for each shutter 110 can be set to default to a shuttered position. Thus, in the event of an operation error, a signal can be sent from the processor 138 to cause the shutters 110 to move to their default position. Furthermore, the shutters 110 can be shuttered as a default until activated (activated via sensing a beverage container 102, which will be discussed later).

The sanitization unit 100 can have at least one UV emitter assembly 108. An exemplary embodiment shows the sanitization unit 100 having four UV emitter assemblies 108. For instance, the sanitization unit 100 can have a UV emitter assembly 108 disposed on an inner surface of the back wall 122, a UV emitter assembly 108 disposed on inner surfaces of each of the two side walls 124, and a UV emitter assembly 108 disposed on an inner surface of a top 130. More or less UV emitter assemblies 108 can be used. The UV emitter assemblies 108 can be orientated in any direction, but it is contemplated for them to be arranged vertically (e.g., the elongated lamps 132 are vertical) when located on the back wall 122 and two side walls 124, and orientated horizontally (e.g., the elongated lamps 132 are horizontal) when located on the top 130. It is contemplated for the beverage container 102 to be place upright within the cradle portion 104 (e.g., the bottom of the beverage container 102 rests upon the plate 112) and thus arranging the UV emitter assemblies 108 in such as fashion will provide the most effective coverage (e.g., allow the UV light emitting therefrom to be incident upon every portion of the beverage container 102).

Similar to the shutter 110 for a UV emitter assembly 108, the housing 120 has a door 106 that selectively covers the opening 128. The arrangement and operation of the door 106 can be similar to that of the shutter 110. The door 106 can be a see-through material with a reflective coating (e.g. transparent or translucent to visible light but blocks UV light). Thus, a user can peer through the door 106 when it is in a shuttered position but still be protected because the UV light will not escape the cradle portion 104 when the door 106 is in the shuttered position. Similar to the shutter 110, the door 106 can have a bias-mechanism to bias the door 106 in a shuttered position. Thus, when the power is turned off or in the event of a power outage, the door 106 automatically shutters the opening 128. In addition, the motor assembly for the door 106 can be set to default to a shuttered position. Thus, in the event of an operation error, a signal can be sent from the processor 138 to cause the door 106 to move to its default position. Furthermore, the door can be shuttered as a default until activated (activated via sensing a beverage container 102, which will be discussed later).

The bottom surface of the housing 120 has the plate 112 or platform upon which the beverage container 102 rests when in use. The plate 112 can be a rotating plate 112. The rotating plate 112 is in mechanical connection with a motor that causes the plate 112 to rotate, thereby causing the beverage container 102 (when placed thereon) to rotate. Thus, after the door 106 is shuttered, the motor can be activated to cause the beverage container 102 to rotate. The UV emitter assembly shutters 110 can then be slid or rotated to un-shutter the UV emitter assemblies 108 and allow the UV light from the UV lamps 132 to be incident upon the beverage container 102 within the cradle portion 104 as the beverage container 102 is rotated via the rotating plate 112. The rotation of the beverage container 102 can ensure that there are no blind spots/locations that do not received a required dosage of UV light, thereby guaranteeing disabling all pathogens on the beverage container 102.

In some embodiments, the plate 112 or platform has illuminators 140 (e.g., LEDs) placed thereon. These illuminators 140 can be used to direct a user in placing the beverage container 102 in the sanitization unit 100. For instance, the illuminators 140 can be in a circular arrangement to inform the user to place the beverage container 102 within the center of the circular arrangement—i.e., placing he beverage container 102 in the center of the circle ensures that the beverage container 102 is centered on the plate 112. Centering the beverage container 102 can further ensure that the UV light being incident upon it hits all portions of the beverage container 102, and further ensure that the beverage container 102 is not toppled over due to rotational moments of the plate 112 when it is rotated. These illuminators 140 can emit light in the visible spectrum. These illuminators 140 can also be illuminated during UV irradiation of the beverage container 102 to give the appearance that that are aiding in the sanitization process.

An exterior portion of the housing 120 can have a sensor 118 (e.g., proximity sensor, RFID reader, etc.) to detect when the beverage container 102 is close (e.g., a predetermined distance from) to the door 106. This can include more than one sensor 118. Upon sensing that a beverage container 102 is in close proximity, a signal is sent to the processor 138 and the processor 138 sends a signal to open (or un-shutter) the door 106, thereby granting access to the cradle portion 104. The processor 138 can include instructions for a fail-safe operations in which the processor 138 will not open the door 106 until it confirms that the shutters 110 for each UV emitter assembly 108 are in a shuttered position. This can be achieved via relay switching, additional sensing (e.g., sensing that the shutter 110 is in a shuttered position, sensing that no or little UV light is within the cradle portion 104, etc.), etc. In addition, or in the alternative, the sanitization unit 100 can have a safety check mechanism in which the door 106 includes an electro-mechanical lock 142. The electro-mechanical lock 142 automatically locks the door 106 in a shuttered position until it independently confirms that the shutters 110 for each UV emitter assembly 108 are in a shuttered position. Again, this can be achieved via relay switching, additional sensing, etc. Thus, regardless of the processor 138 receiving sensing information that the shutters 110 for each UV emitter assembly 108 are in a shuttered position, the electro-mechanical lock 142 can have its own independent sensing system as a redundancy to ensure that the UV emitter assemblies 108 are shuttered. With this embodiment, the door 106 will not un-shutter unless it receives a signal from the processor 138 to open and its electro-mechanical lock 142 inlocks.

As noted above, the operational steps for the sanitization unit 100 are contemplated to be performed automatically. Thus, the door 106 is operated automatically, and a user need not touch the door 106 at all.

In addition, the sanitization unit 100 can include a UV emitter assembly 108 operational sensor 118 (e.g., an ampere sensor) to determine if a UV lamp 132 is operating properly. This can be located inside the housing 120. This can include more than one sensor 118. If any one or combination of UV emitter assemblies 108 are not operating, the processor 138 can receive such a signal and not open (un-shutter) the door 106. Thus, a user would not be able to use the sanitization unit 100 until all (or at least a predetermined number of UV emitter assemblies 108) are operating properly. As will be explained herein, come embodiments of the sanitization unit 100 include a display 114. If any operational error occurs (e.g., a UV emitter assembly 108 not operating), the processor 138 can generate a textual or graphical output via a user interface of the display 114 to notify a user of the error.

In addition, the sanitization unit 100 can include a UV light environment sensor 118 (e.g., a UV sensor) to determine if the UV emitter assemblies 108 are generating enough UV light. This can be located inside the housing 120. This can include more than one sensor 118. Thus, if the UV light being generated is not intense enough (as measured by the UV light environment sensor(s) 118), the processor 138 can receive such a signal and stop operation. At this point, the shutters 110 for each UV emitter assembly 108 resort to their default position (i.e., shutter the windows 136), and the door 106 is open (un-shutter) so as to allow a user to retrieve their beverage container 102.

The sanitization unit 100 can have a sensor 118 (e.g., proximity sensor, RFID reader, etc.) to detect when the beverage container 102 is placed within the cradle portion 104. This can be located inside the housing 120. This can include more than one sensor 118. This can also determine whether the beverage container 102 is centered on the plate 112, whether the beverage container 102 is in an upright position, etc. As noted herein, the sanitization unit 100 is activated by the on/off switch 116 to supply electrical power to all of the operable components. Thus, upon activating the unit 100, the UV emitter assemblies 108 are on and emitting UV light. The shutters 110 are in a shuttered positon for each UV emitter assembly 108 and the door 106 is also shuttered. Upon detecting presence or proximity of the beverage container 102, the door 106 opens. A user places the beverage container 102 within the cradle portion 104 and on the plate 112. Upon sensing that the beverage container 102 is placed on the plate 112, is upright, and is centered on the plate 112, the door 106 is shuttered. The plate 112 begins to rotate and the shutters 110 of the UV emitter assemblies 108 un-shutter to allow UV light to irradiate the beverage container 102. At any time, if the beverage container 102 is detected to be off-centered, toppled over, etc., the processor 138 can receive such a signal and stop operation. At this point, the shutters 110 for each UV emitter assembly 108 resort to their default position (i.e., shutter the windows 136), the plate 112 stops rotating, and the door 106 is open (un-shutter) so as to allow a user to retrieve their beverage container 102.

As noted herein, the door 106 can have an electro-mechanical lock 142 automatically locks the door 106 in a shuttered position. This electro-mechanical lock 142 can include or be in operative association with a sensor 118 that detects whether the shutters 110 for the UV emitter assemblies 108 are shuttered and/or whether there is UV light present within the cradle portion 104. This sensing is independent of the sensing used by the processor 138. Any time, the sensing for the electro-mechanical lock 142 detects an un-shuttered UV emitter assembly 108 or presence of UV light within the cradle portion 104, it can override any operation being carried out that would otherwise unlock and open the door 106. Thus, if the sensing for the electro-mechanical lock 142 detects an un-shuttered UV emitter assembly 108 or presence of UV light within the cradle portion 104, the door 106 will remain shuttered and locked. In addition, if the sensing for the electro-mechanical lock 142 detects an un-shuttered UV emitter assembly 108 or presence of UV light within the cradle portion 104, the door 106 will automatically close or override any door open operation to close and lock the door 106.

The sensor(s) 118 that detect the presence or proximity of the beverage container 102 can be configured to sense the presence or proximity of any object (e.g., a user, a user's hand, a beverage container, another object, etc.). However, the sensor(s) 118 can be more sophisticated and include imaging and image processing techniques (e.g., Gabor filtering, etc.), spectroscopic techniques, etc. to identify certain objects or certain types of objects. Thus, the actuation or activation of operable components can be dependent upon whether a certain type, color, shape, etc. of object is detected. This type of sensing can also detect the type of material of the beverage container 102, the shape and contour of the beverage container 102, etc.

In some embodiments, the sanitization unit 100 can include a sanitization sensor 118. This can be located inside the housing 120. This can include more than one sensor 118. For instance, the sensor 118 can be an optical sensor that detects viruses, bacterial, pathogens, etc. This can be used to determine whether the beverage container 102 has been satisfactorily sanitized. It is contemplated for the sanitization unit 100 to operate for a predetermine amount of time (e.g., 20 seconds) when the beverage container 102 is placed within the unit 100. But, the sanitization sensor 118 can be used to ascertain the level of sanitization obtained for the beverage container 102, and either extend this time or inform a user via the display 114 that an additional sanitization session is required. If the beverage container 102 is detected to not be sanitized to a predetermined level, the processor 138 can receive such a signal and either extend the sanitization time, perform an additional sanitization session, recommend an additional sanitization session, etc.

In addition, the data from the sanitization sensor 118 can be compiled for many sanitization sessions so that statistical analyses can be performed on certain types of beverage containers 102, on the operation of certain components (sensors 118, UV emitter assemblies 108, the unit 100 as a whole, etc.). For instance, the processor 138 can be in operative association with a memory (e.g., a non-transitory non-volatile memory), wherein sanitization data from the sanitization sensor 118 can be stored. The processor 138 can perform analyses on these data and provide the results via the display 114. In addition, or in the alternative, another computer device can be used to acquisition the data from the memory and perform such analyses.

In some embodiments, the sanitization data can be used by the processor 138 to determine a portion of the beverage container 102 that may require additional irradiation or relatively more irradiation than another portion. Thus, the processor 138 can use the sanitization data, as well as other sensing data, to stop or slow the rotation of the plate 112 at certain times or at beverage container 102 rotational position(s) so as to differentiate the irradiation occurring at certain locations on the beverage container 102.

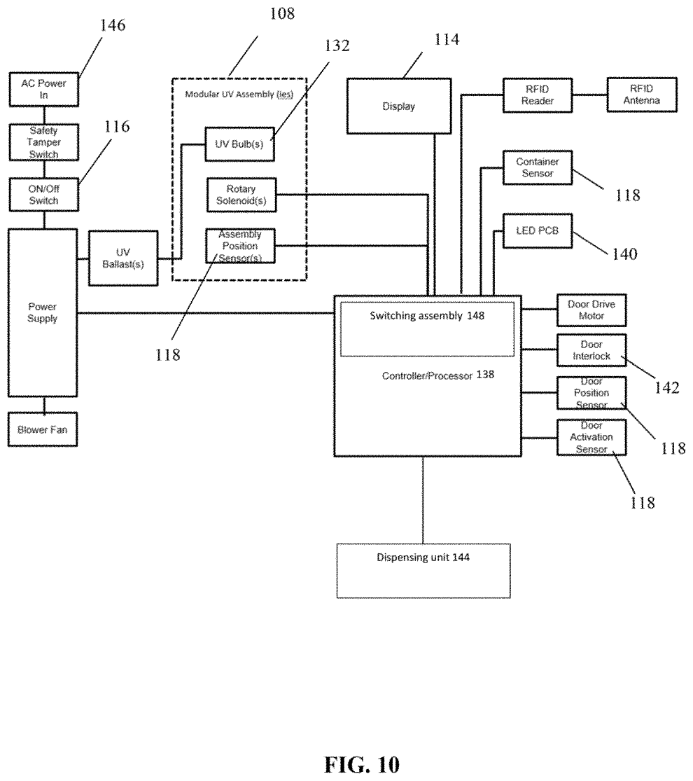

Referring to FIG. 10, with embodiments in which the proximity sensor 118 is a RFID reader, the beverage container 102 can have an RFID tag attached to or embedded therein. This can be done to identify a specific beverage container 102, record and track if and when it has been sanitized, how often it is sanitized, etc. In some embodiments, the sanitization unit 100 can be in operative association with a dispensing unit 144 (e.g., a unit that dispenses beverage). Being in operative association can include being integral to the dispensing unit 144, being in communication (e.g., hardwired or wireless) with the dispensing unit 144, etc. The sanitization data of any one or combination of beverage containers 102 can be transmitted from the processor 138 to a control module of the dispensing unit 144. This can be done to discriminatorily allow disbursement of beverage from the dispensing unit based on the sanitization of the beverage container 102—i.e., the dispensing unit 144 may not dispense beverage unless it receives information that the beverage container 102 has been sanitized. For instance, the dispensing unit 144 may also include a RFID reader and require a user to scan the beverage container 102. The dispensing unit 144 can then acquisition sanitization data from the processor 138 to ascertain if/when the beverage container has been sanitized. If it has not been sanitized or has not been sanitized within a certain amount of time before attempting to be used to receive beverage from the dispensing unit 144, the dispensing unit 144 can prevent disbursement of beverage. The dispensing unit 144 can also include a display and may inform the user via the display that no beverage will be dispensed for that particular beverage container 102.

It should be noted that any one or combination of the operable components (e.g, motors, sensors 118, UV emitter assembly 108, etc.) of the sanitization unit 100 can be in electrical communication with a processor 138 and an electrical power supply 146. For instance, any one or combination of the operable components can be in electrical connection with a switching assembly 148 (e.g., a multiplexer). The switching assembly 148 can be in electrical connection with both the electrical power supply 146 (via the on/off switch 116) and the processor 138. The processor 138 can be in operative association with a memory that has software instructions stored thereon. The processor 138 can operate in accordance with the software instructions to control the operable components via the switching assembly 148 so as to implement any of the methods disclosed herein. As noted herein, the processor 138 may be in communication with a computer device, a control module of a dispensing unit 144, etc. This can be via a hardwire connection or a wireless connection.

The top 130 of the housing 120 can include a display 114. The display 114 can include a display screen for displaying a user interface or other graphical interface. The display 114 may include the processor 138 and associated memory, as other processors and circuitry to facilitate command and control of the sanitization unit 100 via the user interface. The user interface can also display statistical sanitization data about a beverage container 102, display a timer to indicate the time left in a sanitization session, data from the sanitization sensor 118, whether the door 106 is locked, whether the beverage container 102 has been sanitized to an acceptable level, whether an additional sanitization session is recommended, etc. If the sanitization unit 100 is integral with the dispensing unit 144, this display may be shared by both units.

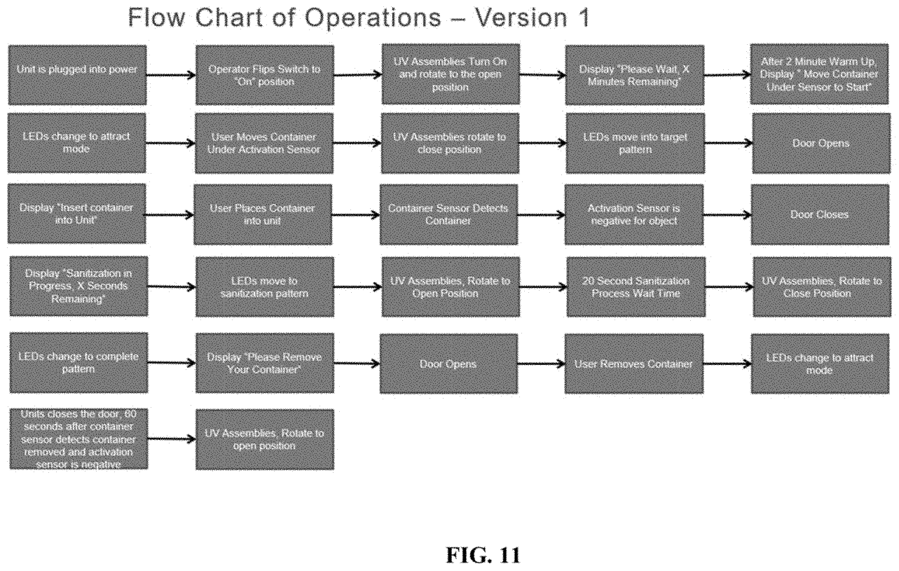

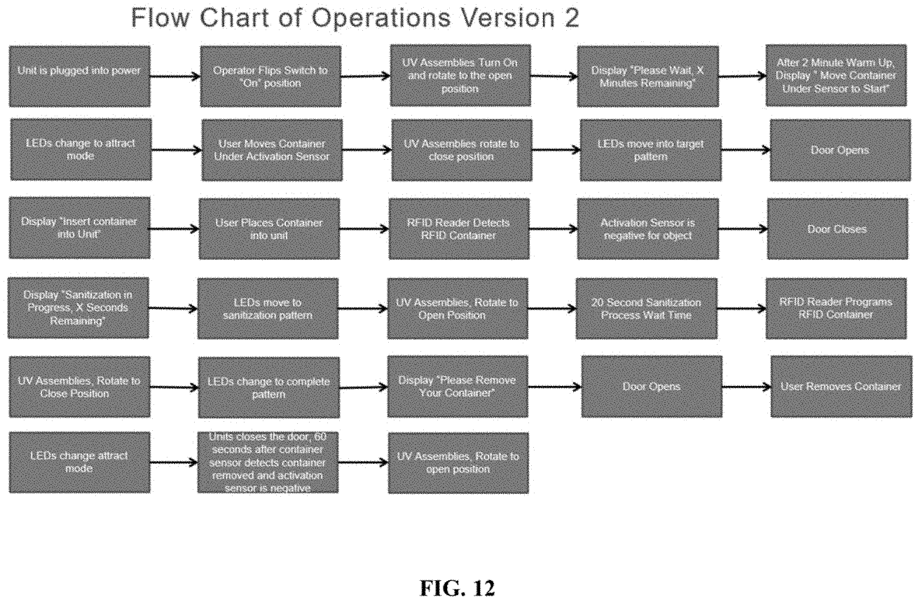

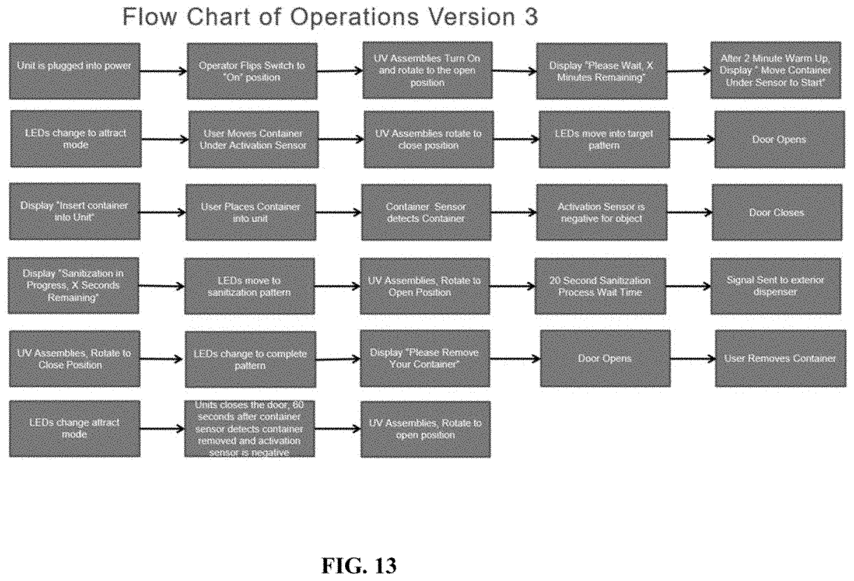

FIGS. 11-13 show exemplary flow diagrams for operating embodiments of the UV sanitization unit 100.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

What is claimed is:

1. A sanitization unit, comprising:
a housing having at least one ultraviolet emitter assembly, the housing configured to have a cradle portion to receive an object;
a door comprising a material that is transparent or translucent to light in the visible spectrum but blocks light in the ultraviolet spectrum, the door further comprising an actuator to open and close the door;
a processor in communication with a sensor, wherein:
the sensor detects presence or proximity of the object, and emits a signal;
the processor receives the signal and, upon receipt of said signal, automatically actuates the door actuator to grant access to the cradle portion.

2. The sanitization unit of claim 1, comprising:
a rotating plate upon which the object is placed when placed within the housing, the rotating plate further comprising an actuator to rotate the plate.

3. The sanitization unit of claim 2, comprising:
a sensor to detect when the object is placed on the plate, wherein when detection occurs the sensor emits a signal;
wherein the processor receives the signal and, upon receipt of said signal, automatically actuates the plate actuator to rotate the plate.

4. The sanitization unit of claim 1, wherein:
the at least one ultraviolet emitter assembly comprises an ultraviolet lamp.

5. The sanitization unit of claim 1, wherein:
the at least one ultraviolet emitter assembly comprises a shroud configured to selectively shutter the at least one ultraviolet emitter assembly.

6. The sanitization unit of claim 5, wherein:
upon activation of the sanitization unit, the at least one ultraviolet emitter assembly continuously emits ultraviolet light.

7. The sanitization unit of claim 1, further comprising:
a sensor to detect when the object is placed within the cradle portion, wherein when detection occurs the sensor emits a signal;
wherein:
the at least one ultraviolet emitter assembly comprises a shroud configured to selectively shutter the at least one ultraviolet emitter assembly;
upon activation of the sanitization unit, the at least one ultraviolet emitter assembly continuously emits ultraviolet light;
when the processor receives the signal, the processor causes the shroud for the at least one ultraviolet emitter assembly to automatically un-shutter the at least one ultraviolet emitter assembly.

8. The sanitization unit of claim 1, further comprising:
a sensor to detect shape, color, material composition, and/or orientation of the object.

9. The sanitization unit of claim 1, further comprising:
a sensor to configured as a RFID reader; and
wherein the object includes a RFID tag.

10. The sanitization unit of claim 1, further comprising:
a sanitization sensor.

11. The sanitization unit of claim 1, further comprising:
an ultraviolet light environment sensor.

12. The sanitization unit of claim 1, wherein:
the door includes an electro-mechanical lock.

13. The sanitization unit of claim 1, further comprising:
the door includes an electro-mechanical lock;
wherein the processor is in connection with first ultraviolet light environment sensor and the electro-mechanical lock is in connection with a second ultraviolet light environment sensor.

14. The sanitization unit of claim 1, further comprising:
a display.

15. The sanitization unit of claim 14, wherein:
the display is configured to generate a user interface that facilitates command and control of the sanitization unit.

16. The sanitization unit of claim 1, further comprising:
a display; and
a sanitization sensor;
wherein the sanitization sensor generates sanitization data that is displayed via a user interface of the display.

17. A sanitization and dispensing system, comprising:
sanitization unit configured to receive a beverage container and sanitize said beverage container via ultraviolet radiation; and
a beverage dispensing unit configured to disburse beverage;
wherein the sanitization unit is in communication with the beverage dispensing unit.

18. The sanitization and dispensing system of claim 17, wherein:
the sanitization unit tracks sanitization data pertaining to the beverage container and transmits said sanitization data to the beverage dispensing unit.

19. The sanitization and dispensing system of claim 18, wherein:
the beverage dispensing unit does not disburse beverage unless said sanitization data indicates that the beverage container has been sanitized.

20. The sanitization and dispensing system of claim 18, wherein:
the beverage dispensing unit does not disburse beverage unless said sanitization data indicates that the beverage container had been sanitized within a predetermine time before the beverage dispensing unit receives said sanitization data.
